(19) United States
(12) Patent Application Publication
(10) Pub. No.: US 2013/0122113 A1
(43) Pub. Date: May 16, 2013

(54) ANTITUMORAL COMBINATION COMPRISING OMBRABULIN, A TAXANE DERIVATIVE AND A PLATINUM DERIVATIVE

(71) Applicant: SANOFI, Paris (FR)

(72) Inventors: Patrick COHEN, Paris (FR); Ileana Corina OPREA, Paris (FR)

(73) Assignee: SANOFI, Paris (FR)

(21) Appl. No.: 13/718,335

(22) Filed: Dec. 18, 2012

Related U.S. Application Data
(63) Continuation of application No. PCT/IB2011/052628, filed on Jun. 16, 2011.

(30) Foreign Application Priority Data
Jun. 18, 2010 (EP) 10305653.7
Nov. 15, 2010 (EP) 10306256.8

(51) Int. Cl. A61K 31/167; A61K 31/337; A61K 31/555; A61K 33/24

(52) U.S. Cl. CPC A61K 31/167; A61K 33/24; A61K 31/337; A61K 31/555
USPC 424/649; 514/449

(57) ABSTRACT

The invention concerns an antitumoral combination comprising ombrabulin, a taxane derivative and a platinum derivative and its use in the treatment of advanced solid tumors.

The invention concerns an antitumoral combination comprising ombrabulin, a taxane derivative and a platinum derivative and its use in the treatment of advanced solid tumors.

### PRIOR ART AND PROBLEM TO BE SOLVED

WO 99/51246 discloses the ombrabulin/platinum salt combination.

WO 2004/037258 discloses the combination of ombrabulin with various antitumoral agents including taxanes (Taxol®, Taxotere®).

There is still a need to find and optimize new therapeutic options to treat patients with advanced solid tumors.

The invention meet this need by providing a new pharmaceutical antitumoral combination comprising ombrabulin, a taxane derivative and a platinum derivative for which doses of each component and a suitable administration protocol has been determined, to obtain a well tolerated combination which does not exacerbate the toxicity of each of the antitumoral agents and which allows the treatment of advanced solid tumors either by stabilizing or by inducing a partial or a complete regression of the tumor.

### DESCRIPTION OF THE INVENTION

The invention concerns an antitumoral combination comprising ombrabulin, a taxane derivative and a platinum derivative, these therapeutic components being in the form of a free base or of an addition salt with a pharmaceutical acceptable acid, or in the form of a hydrate or of a solvate, where this antitumoral combination is well tolerated, does not exacerbate the toxicity of each of the antitumoral agents and which allows the treatment of advanced solid tumors either by stabilizing or by inducing a partial or a complete regression of the tumor.

Ombrabulin (AVE8062) belongs to the family of combretastatins and has the formula:

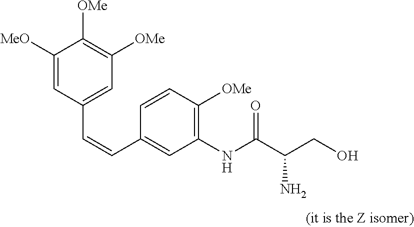 [SMILES: COc1ccc(/C=C\c2cc(OC)c(OC)c(OC)c2)cc1NC(=O)[C@@H](N)CO]

It is an antivascular agent (or VDA, Vascular Disrupting Agent). It has the chemical name: (Z)-N-[2-methoxy-5-[2-(3,4,5-trimethoxyphenyl)vinyl]phenyl]-L-serinamide.

This compound, which is described in EP 731085 B1, may be prepared according to the method described in WO 03/084919. Ombrabulin may be administered in base form (cf. above formula) or in the form of a salt of a pharmaceutically acceptable acid, for example in the form of the hydrochloride, represented below:

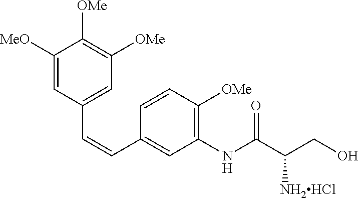

Once administered, ombrabulin releases in vivo the active metabolite (Z)-1-(3-amino-4-methoxyphenyl)-2-(3,4,5-trimethoxyphenyl)ethene, which has the formula:

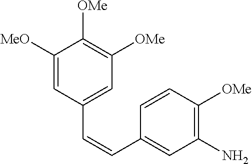 [SMILES: COc1ccc(/C=C\c2cc(OC)c(OC)c(OC)c2)cc1N]

It is therefore also possible to substitute, for ombrabulin, another combretastatin of formula:

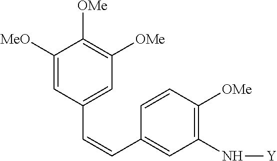

in base form or in the form of a salt of a pharmaceutically acceptable acid, in which Y represents an amino acid, which releases in vivo this metabolite.

The taxane derivative may for example be chosen from paclitaxel or docetaxel.

The platinum derivative may for example be chosen from cisplatin or carboplatin.

The combination comprises an effective quantity of ombrabulin, an effective quantity of a taxane derivative and an effective quantity of a platinum derivative.

Ombrabulin may be administered by perfusion at a dose comprised between 15 and 35 mg/m², for example chosen from the following doses: 15.5; 20; 25; 30 and 35 mg/m².

Docetaxel may be administered by perfusion at a dose of 60 or 75 mg/m².

Paclitaxel may be administered by perfusion at a dose of 175 or 200 mg/m².

Cisplatin may be administered by perfusion at a dose of 75 mg/m².

Carboplatin may be administered by perfusion at a dose of AUC 5 and AUC 6.

Preferentially, ombrabulin may be used in combination with docetaxel and cisplatin or in combination with paclitaxel and carboplatin.

Preferentially, ombrabulin may be used in combination with docetaxel and cisplatin.

In this case, ombrabulin may be administered at a dose of 20 mg/m², docetaxel at a dose of 75 mg/m² and cisplatin at a dose of 75 mg/m².

In this case, ombrabulin may also be administered at a dose of 35 mg/m², docetaxel at a dose of 75 mg/m² and cisplatin at a dose of 75 mg/m².

Preferentially, ombrabulin may be used in combination with paclitaxel and carboplatin.

In this case, ombrabulin may be administered at a dose of 35 mg/m², paclitaxel at a dose of 175 mg/m² and carboplatin at a dose of 5 AUC.

In this case, ombrabulin may also be administered at a dose of 35 mg/m², paclitaxel at a dose of 200 mg/m² and carboplatin at a dose of 6 AUC.

The cycle of administration of the three antitumoral agents is repeated with an interval between two administrations of three weeks.

The invention also concerns the use of ombrabulin, a taxane derivative and a platinum derivative for the preparation of an antitumoral combination here above disclosed.

The invention also concerns the above disclosed antitumoral pharmaceutical combination comprising ombrabulin, a taxane derivative and a platinum derivative, these agents being in the form of a free base or of an addition salt with a pharmaceutical acceptable acid, or in the form of a hydrate or of a solvate, for its use as a medicament in the treatment of advanced solid tumors.

The invention also concerns a method of treating advanced solid tumors in a patient in need thereof, said method comprising administrating to said patient therapeutically effective amounts of the above disclosed antitumoral pharmaceutical combination comprising ombrabulin, a taxane derivative and a platinum derivative, these agents being in the form of a free base or of an addition salt with a pharmaceutical acceptable acid, or in the form of a hydrate or of a solvate.

Examples of solid tumors that may be treated with the combination of the invention are—but not exclusively—lung tumors, ovarian tumors and breast tumors including triple negative breast tumors.

In another aspect the invention provides for an article of manufacture comprising:
a packaging material
the above disclosed antitumoral pharmaceutical combination comprising ombrabulin, a taxane derivative and a platinum derivative, these agents being in the form of a free base or of an addition salt with a pharmaceutical acceptable acid, or in the form of a hydrate or of a solvate, and
a label or package insert contained within said packaging material indicating that said antitumoral pharmaceutical combination is administered to the patient at a recommended dose, and in a plurality of subsequent doses at a recommended dose separated in time from each other by three weeks.

The recommended doses are as described in the following study.

Definitions
pharmaceutically acceptable acid: organic or inorganic acid having a low toxicity (see “Pharmaceutical salts” J. Pharm. Sci. 1977, 66, 1-19);
effective amount: amount of a pharmaceutical compound that produces an effect on the treated tumour.
   advanced solid tumors: locally advanced or metastatic solid tumors ie tumors which are not operable any more.

The combination is administered repeatedly in a course of several cycles according to a protocol that depends on the nature and on the stage of the cancer to be treated and also on the patient to be treated (age, weight, previous treatment(s), etc.).

Examples of cycles and doses are given in the study below.

An open-label, non-randomized, dose escalation, safety and pharmacokinetics phase I study of ombrabulin in combination with platinum salts (cisplatin or carboplatin) and taxanes (docetaxel or paclitaxel), every 3 weeks, in patients with advanced solid tumors has been done.

Study Objective(s)

Primary Objective

The primary objective of the study is to determine the recommended dose (RD) based on the incidence of dose limiting toxicity (DLT), the maximum administered dose (MAD), and the maximum tolerated dose (MTD) of ombrabulin in combination with platinum salts and taxanes, every 3 weeks in patients with advanced solid tumors for which platinum-taxane doublet has been approved or constitutes mainstay of care.

Thus, the primary endpoint of the study is:

Dose Limiting Toxicity (DLT) at cycle 1.

Secondary Objectives

The secondary objectives of the study are:

To assess the overall safety profile of the combination.

To characterize at Cycle 1 the pharmacokinetic (PK) profile of ombrabulin given with platinum salts and taxanes following different schedules.

To evaluate anti-tumor activity of the tritherapy combination.

To evaluate potential predictive biomarkers.

Thus, the secondary endpoints of the study are:

TEAE (Treatment Emergent Adverse Event), post-TEAE, SAE (Serious Adverse Event) and laboratory abnormalities.
At cycle 1: pharmacokinetic (PK) parameters of ombrabulin given with platinum salts and taxanes following different schedules.
Objective tumor response as defined by the RECIST criteria.

Study Design

Two groups of patients will be treated: one with docetaxel-cisplatin doublet (group 1) and the second with paclitaxel-carboplatin doublet (group 2), both in combination with ombrabulin.

The combination will be started with the following schedule (schedule A) for group 1:

Day 1: ombrabulin as a 30 minutes i.v. infusion immediately followed by 120 minutes i.v. infusion of cisplatin, and

Day 2: docetaxel administered as a 60 minutes i.v. infusion 24 hours apart ombrabulin infusion end and for the first 4 dose levels (I, II, III, IV).

Cohorts of 3 or 6 patients will receive escalating doses of ombrabulin (15.5, 20 and 25 mg/m²) with a fixed dose of cisplatin at 75 mg/m² on Day 1, followed by docetaxel on Day 2, given either at 60 mg/m² for the ombrabulin doses of 15.5 and 20 mg/m² or at 75 mg/m² for the ombrabulin doses of 20 and 25 mg/m².

Taking into consideration the recommended dose of the combination ombrabulin and cisplatin administered the same day (25 mg/m² and 75 mg/m² respectively) every 3 weeks, after dose level IV, even if MAD not reached at this dose level, dose escalation of ombrabulin will be stopped and the combination will be administered with the following schedule (schedule B) in the 2 groups:

Day 1: ombrabulin as a 30 minutes i.v. infusion, and

Day 2:

Group 1: docetaxel administered as a 60 minutes i.v. infusion followed by cisplatin as a 120 minutes i.v. infusion, 24 hours apart ombrabulin infusion end.

Group 2: paclitaxel administered as a 180 minutes i.v. infusion followed by carboplatin as a 30 minutes i.v. infusion, 24 hours apart ombrabulin infusion end.

At each level, cohorts of 3 or 6 patients will receive escalating doses of ombrabulin (20, 25, 30, 35 . . . mg/m²) followed at Day 2 by a fixed dose of cisplatin at 75 mg/m² or carboplatin AUC 5 or 6 in combination with docetaxel given at 75 mg/m² or paclitaxel either at 175 (regimen A) or 200 mg/m² (regimen B).

Group 1 (ombrabulin/docetaxel/cisplatin):

Dose escalation: ombrabulin with cisplatin
(CDDP) and docetaxel (TXT)

| | Dose- | ombrabulin | CDDP | TXT |
|---|---|---|---|---|
| | Levels | mg/m² | mg/m² | mg/m² |
| Schedule A | I | 15.5 | 75 | 60 |
| (ombrabulin | II | 20 | 75 | 60 |
| and CDDP | III | 20 | 75 | 75 |
| Day 1, TXT | IV | 25 | 75 | 75 |
| Day 2) | | | | |
| Schedule B | V* | 20 | 75 | 75 |
| (ombrabulin | VI | 25 | 75 | 75 |
| Day 1, CDDP | VII | 30 | 75 | 75 |
| and TXT Day 2) | VIII | 35 | 75 | 75 |

*if 2 DLTs at this dose level, possibility to test ombrabulin at 15.5 mg/m² - docetaxel 75 mg/m² - cisplatin 75 mg/m²

Group 2 (ombrabulin/paclitaxel/carboplatin):

Dose escalation: ombrabulin with carboplatin
(Cb) and paclitaxel (PXL): Regimen A

| | Dose- | ombrabulin | Cb | PXL |
|---|---|---|---|---|
| | Levels | mg/m² (D1) | AUC (D2) | mg/m² (D2) |
| Schedule B | Ia* | 20 | 5 | 175 |
| (ombrabulin | Ia′ | 20 | 6 | 175 |
| Day 1, Cb and | IIa | 25 | 5 | 175 |
| PXL Day 2) | IIIa | 30 | 5 | 175 |
| | IVa | 35 | 5 | 175 |

*if 2 DLTs at this dose level, possibility to test ombrabulin at 15.5 mg/m²

In group 2, dose escalation could be continued by increasing ombrabulin of 20% from previous dose for a maximum of 50 mg/m² (which is the recommended dose of the drug in monotherapy), provided that tested dose levels had not shown 2 or more DLTs.

In group 1, dose escalation will be stopped after dose level 35 mg/m² for ombrabulin, taking into account the recommended dose that has been reached with the bi-therapy (ombrabulin 35 mg/m² and docetaxel 75 mg/m²) in an on-going phase I trial.

Patients will then be followed for 21 days for safety assessment. After at least 21 days, patients will receive additional courses at every 21-day intervals in the absence of disease progression, unacceptable toxicity, or other study treatment criteria.

Thus, a cycle is defined as a 3 week-period including one ombrabulin, platinum salt and taxane administration.

Recruitment in groups 1 and 2 could be run in parallel. The first dose levels to be tested in group 2 will be:
ombrabulin 20 mg/m²—Carboplatin AUC 5—Paclitaxel 175 mg/m² (dose level Ia), followed by
ombrabulin 20 mg/m²—Carboplatin AUC 6—Paclitaxel 175 mg/m² (dose level Ia′) followed by
ombrabulin 20 mg/m²—Carboplatin AUC 6—Paclitaxel 200 mg/m² (dose level Ib)

Then dose levels IIa-IIIa-IVa (regimen A) and IIb-IIIb-IVb (regimen B) could be run in parallel.

Once the MAD is reached in each group and regimen with schedule B, additional patients to complete a subset of at least 15 patients, will be treated at the immediate lower dose of ombrabulin with both platinum-taxane doublets chemotherapy (MTD) schedule B, mainly patients with non small cell lung cancer and ovarian cancer.

Dose escalation: ombrabulin with carboplatin
(Cb) and paclitaxel (PXL): Regimen B

| | Dose- | ombrabulin | Cb | PXL |
|---|---|---|---|---|
| | Levels | mg/m² (D1) | AUC (D2) | mg/m² (D2) |
| Schedule B | Ib | 20 | 6 | 200 |
| (ombrabulin | IIb | 25 | 6 | 200 |
| Day 1, Cb and | IIIb | 30 | 6 | 200 |
| PXL Day 2) | IVb | 35 | 6 | 200 |

NB: The first dose level to be tested in group 2 will be Ia, followed by Ia′ then Ib. Then dose levels IIa-IIIa-IVa and IIb-IIIb-IVb could be run in parallel; dose escalation could be continued by increasing ombrabulin of 20% from previous dose, provided that tested dose levels had not shown 2 or more DLTs

Cohorts of 3 or 6 patients will be screened and treated at each dose level. When the first three patients of a cohort have completed the first cycle, i.e. should have received at least one treatment course and should have been observed for acute toxicity for at least a 3-week follow-up period (or shorter period provided that a DLT has been observed), dose escalation strategy will be as follows:

In the absence of DLT at first cycle, three patients will be treated at the next dose level.

If DLT is observed at first cycle in 1 out of 3 patients, three further patients will be included at the same dose level and possibly at the same time.

Then, if DLT is observed at first cycle in 1 out 6 patients, the next dose level will be tested. Otherwise, if 2 out 6 patients present with a DLT at first cycle, the MAD is considered to be achieved.

If DLT is observed at first cycle in 2 out of the 3 patients, the MAD is considered to have been reached.

The Maximum Administered Dose (MAD) will be reached at the dose at which ≧2 out of 3-6 patients develop a DLT at the first cycle.

The dose limiting toxicities (DLTs) that are events to be watched and which allow to drive the escalation of dose, were predefined in the protocol in agreement with the scale of classification NCl-CTCAE version 3.

Route(s) of administration:

ombrabulin, cisplatin, carboplatin, paclitaxel and docetaxel will be administered by intravenous infusion

Study Population

Main Inclusion Criteria
Advanced neoplastic disease (i.e. metastatic or locally advanced disease) for which platinum-taxane doublet regimens are approved or constitutes the mainstay of care such as non small cell lung cancer, epithelial ovary cancer, gastric cancer, transitional cell and bladder cancer and head and neck cancer.
First or second line of metastatic disease.
≧18 years old.
ECOG performance status of 0 to 1.
No brain metastase or carcinomatous leptomeningitis.
No peripheral neuropathy grade >1.

Main Exclusion Criteria
Related to the Methodology (concurrent treatment with any other anticancer therapy, absence of histologically or cytologically proven cancer at the first diagnosis, washout period of less than 3 weeks from prior anti-tumor therapy like chemotherapy, targeted agents, immunotherapy and radiotherapy or any investigational treatment, of less than 6 weeks from prior therapy with nitrosoureas or mitomycin).
Related to the study drugs (previous carboplatin dose higher than 3000 mg/m² or cisplatin higher than 600 mg/m²; more than 1 line of previous chemotherapy as treatment for advanced cancer disease, neoadjuvant treatment excluded; severe hypersensitivity due to taxanes, polysorbate 80 and any other compound of the study combination; unadequate organ function including: neutrophils <1.5×109/L; platelets <100×109/L; creatinine≧1.5 mg/dl, total bilirubin not within normal limit and ALT/AST/AP>2.5 times the upper normal limits of the institutional norms).
Cardiovascular exclusion criteria (documented medical history of myocardial infarction, documented angina pectoris, arrhythmia especially severe conduction disorder such as second or third-degree atrio-ventricular block, stroke, or history of arterial or venous thrombo-embolism within the past 6 months requiring anticoagulants; patient with a LVEF<50% by echocardiography; patient with uncontrolled hypertension and patient with organ damage related to hypertension such as left ventricular hypertrophy or grade 2 ocular funduscopic changes or kidney impairment; 12-lead ECG: infarction Q-wave (at least in 2 contiguous derivations, duration >40 msec, amplitude >20% of QRS complex), ST segment depression or elevation ≧1 mm in at least 2 contiguous leads; untreated hypertension defined as systolic BP>140 mmHg or diastolic BP>90 mmHg on two repeated measurements at 30 minutes interval).

Results:

T (docetaxel D or paclitaxel P) and PS (cisplatin C or carboplatin Cb respectively)

Ob=ombrabulin

pt=patient; pts=patients

d=day

Sixty-nine patients (23 males and 46 females), median age 49 (range 24-74), including 21 chemonaive patients, were treated in 4 cohorts:
Cohort I (Ob/C75 mg/m²d1, D60 or 75 mg/m² d2—13 pts)
Cohort II (Ob d1, C75/D75 d2—19 pts)
Cohort III (Ob d1, CbAUC5/P175 d2—18 pts)
Cohort IV (Ob d1, CbAUC6/P200 d2—19 pts).

Dose levels (DL) tested for Ob were: 15.5, 20, 25, 30, 35 mg/m².

Granulocyte growth factors were systematically administered as primary prophylaxis in cohort I and II.

The most common tumor types were lung (n=14), breast (n=19, including 5 triple negative pts) and ovarian (n=9).

Concerning Cohort I:
the median number of cycles was 6 (range 1-16);
the RD is Ob20/C75/D75 mg/m².

Two DLTs (febrile neutropenia and grade 4 pulmonary embolism) were reported at cycle 1 of dose levels 25/75/75 mg/m².

The most frequent TEAEs were: asthenia (12 pts including 1 grade 3), nausea (11 pts), paresthesia (10 pts), diarrhea (7 pts including 1 grade 3). Other related grade ¾ TEAEs were: 1 grade 3 drug hypersensitivity. Related cardiovascular events consisted on: grade 2 thrombo-phlebitis (2 pts), grade 1 sinusal bradycardia (1 pt), grade 2 deep venous thrombosis (1 pt) and grade 1 orthostatic hypotension (1 pt).

Hematotoxicity was typical for D and C combinations.

Objective responses were observed: on 11 evaluable pts, there were 4 partial responses (including 1 epidermoid lung cancer).

Concerning Cohort II
the median number of cycles was 6 (range 2-15);
the maximum administered dose was reached at 35 mg/m² for ombrabulin;
the RD is Ob35/C75/D75 mg/m²; only 1 DLT (grade 3 transaminase increase) was observed at the first dose level (20/75/75)

The most frequent TEAEs were: asthenia (19 pts, including 1 grade 3), nausea (17 pts), paresthesia (13 pts), stomatitis (10 pts), vomiting (12 pts), alopecia (13 pts). Other related grade ¾ TEAEs were 1 grade 3 drug hypersensitivity and 2 grade 3 pulmonary embolism. Related cardiovascular events not listed as grade ¾ consisted on: grade 2 hypertension (1 pt), grade 1 orthostatic hypotension (1 pt) and grade 2 LVEF decrease (1 pt).

Hematotoxicity was typical for D and C combination.

Objective responses were observed: on 18 evaluable pts, 6 partial responses (2 lung including 1 epidermoid lung cancer, 2 breast and 1 uterus cancer) were obtained.

Concerning Cohort III
the median number of cycles was 2 (range 1-8);
the maximum administered dose was reached at 35 mg/m² for ombrabulin;
the RD is Ob35 mg/m²/Cb5 AUC /P175 mg/m²; no DLT was observed.

The most frequent TEAEs were: asthenia (16 pts), alopecia (13 pts), vomiting (12 pts), nausea (11 pts), paresthesia (11 pts) and stomatitis (9 pts). Related grade ¾ TEAEs were: 1 grade 3 drug hypersensitivity. Related cardiovascular events consisted on: grade 3 hypertension (1 pt).

Hematotoxicity was typical for P and Cb combination.

Objective responses were observed: on 17 evaluable pts, 1 complete response (triple negative breast cancer) and 2 partial responses (ovarian lung cancer) were obtained.

Concerning Cohort IV
the median number of cycles was 4 (range 1-12);
the maximum administered dose was reached at 35 mg/m² for ombrabulin;
the RD is Ob35 mg/m² /Cb6 AUC /P200 mg/m²;
only 1 DLT was observed: grade 3 toe ischemia at the first dose level tested (Ob20 mg/m²/Cb6 AUC /P200 mg/m²).

The most frequent TEAEs were: decrease apetite (11 pts), vomiting (10 pts), asthenia (17 pts including 1 grade 3), nausea (11 pts including 1 grade 3), alopecia (11 pts) and paresthesia (15 pts).

Other related grade ¾ TEAEs were: 1 grade 3 peripheral neuropathy. Related cardiovascular events consisted on: grade 1 sinusal bradycardia (1 pt), grade 2 hypertension (2 pts).

Hematotoxicity was typical for P and Cb combination.

Objective responses were observed: on 18 evaluable pts, 3 partial responses (lung, ovary and thymoma).

Thus, these results confirm that the combination of Ob with T and PS is feasible and well tolerated, with preliminary encouraging evidence of anti-tumor activity.

Pharmacokinetic Study

Blood samples for pharmacokinetic analysis were obtained from all patients on Day 1, 2 and 3 at Cycle 1.

Group 1

AVE8062

A series of 2-mL blood samples were collected in heparinized (lithium heparinate) tubes as follows:
immediately prior to the end of infusion;
at 5, 10, 25, 45 and 60 minutes post infusion;
at 2, 4, 6, 8, 10 and 24 hours post infusion (i.e a total of 24 mL of blood).

Cisplatin

A series of 5-mL blood samples were collected in heparinized (sodium heparinate) tubes as follows:
immediately prior to the end of infusion;
at 30 and 60 minutes post infusion;
at 2, 4, 6, 8 and 22 hours post infusion (i.e a total of 40 mL of blood).

Docetaxel

A series of 2-mL blood samples were collected in heparinized (lithium heparinate) tubes as follows:
15 minutes before the end of docetaxel infusion;
at 15 and 45 minutes post docetaxel infusion;
at 2 and 5 hours post docetaxel infusion (i.e. a total of 10 mL of blood).

Group 2

AVE8062

A series of 2-mL blood samples were collected in heparinized (lithium heparinate) tubes as follows:
immediately prior to the end of infusion;
at 5, 10, 25, 45 and 60 minutes post infusion;
at 2, 4, 6, 8, 10 and 24 hours post infusion (i.e a total of 24 mL of blood).

Paclitaxel

A series of 2-mL blood samples were collected in EDTA tubes as follows
90 minutes and immediately prior to the end of infusion
at 0.5, 1, 2, 4, 6, 8 and 24 hours post infusion (i.e. a total of 18 mL of blood)

Carboplatin

A series of 3-mL blood samples were collected as follow:
immediately prior to the end of infusion;
at 0.5, 1.5, 3.5, 5.5, 7.5 and 23.5 hours post infusion (i.e. a total of 21 mL of blood)

Results :

Ombrabulin clearance was high (72.9 L/h/m²) and the volume of distribution at steady state was small (25.0 L/m²), corresponding to a short terminal elimination half-life (17 min).

Ombrabulin was rapidly converted to its active metabolite which has a terminal elimination half-life of around 11 h.

Metabolite exposure was found to be about 2-fold higher than ombrabulin.

The table 1 shows mean ombrabulin pharmacokinetic parameters at cycle1.

The table 2 shows mean ombrabulin metabolite pharmacokinetic parameters at cycle1.

TABLE 1
Mean ombrabulin pharmacokinetic parameters at cycle 1.

| Dose(Ombrabulin) | | tₘₐₓ* | Cₘₐₓ | AUC₍₀₋ₜ₎ | AUC | t_{1/2Z} | CL | Vₛₛ |
|---|---|---|---|---|---|---|---|---|
| (mg/m²) | Nb of patients | (h) | (ng/mL) | (ng · h/mL) | (ng · h/mL) | (min) | (L/h/m²) | (L/m²) |
| 15.5 | 3 | 0.48 [0.47-0.48] | 712 (30) | 274 (29) | 276 (29) | 20.9 (76) | 602 (34) | 18.2 (32) |
| 20 | 26 | 0.48 [0.42-0.73] | 861 (38) | 351 (36) | 353 (36) | 17.7 (50) | 64.6 (42) | 22.0 (57) |
| 25 | 12 | 0.49 [0.42-0.63] | 973 (46) | 381 (45) | 383 (45) | 15.3 (37) | 80.1 (49) | 27.2 (76) |
| 30 | 6 | 0.54 [0.48-1.0] | 1170 (37) | 508 (27) | 510 (27) | 18.5 (65) | 63.4 (33) | 24.3 (37) |
| 35 | 5 | 0.5 [0.48-0.5] | 1270 (55) | 491 (57) | 493 (57) | 14.3 (28) | 95.7 (63) | 32.8 (78) |
| Overall | 52 | | | | | 17.3 (15) | 72.9 (23) | 25.0 (33) |

*Median and range are presented instead of mean and CV

TABLE 2
Mean ombrabulin metabolite pharmacokinetic parameters at cycle1.

| Dose(Ombrabulin) | | Cₘₐₓ | AUC₍₀₋ₜ₎ | AUC | t_{1/2Z} | |
|---|---|---|---|---|---|---|
| (mg/m²) | Nb of patients | (ng/mL) | (ng · h/mL) | (ng · h/mL) | (min) | Metabolic Ratio |
| 15.5 | 3 (1^{a}) | 145 (17) | 783 (70) | 519 | 13.1 | 3.52 (100) |
| 20 | 26 (23^{a}) | 166 (14) | 425 (29) | 465 (26) | 9.22 (40) | 1.52 (40) |
| 25 | 12 (12^{a}) | 192 (16) | 461 (17) | 518 (18) | 9.41 (33) | 1.61 (45) |
| 30 | 6 (5^{a}) | 252 (19) | 628 (20) | 661 (23) | 8.79 (29) | 1.38 (27) |
| 35 | 5 (5^{a}) | 264 (28) | 623 (26) | 798 (35) | 13.9 (48) | 2.17 (78) |
| Overall | 52 (46^{a}) | | | | 10.9 (22) | 2.04 (43) |

^{a}Number of patients reported for AUC and t_{1/2Z} calculation

Biomarkers Study

Tumor biopsies were performed on 11 patients, immunohistochemical and RT-PCR methods were used.

On 11 patients, 3 had high score for CD31 (ovary, uterus and liver cancer), 9 for CD34 (mainly ovarian, breast, liver cancer) and 1 for CD 105 (ovarian cancer). All cases were stained in intratumoral vessels, indicating that these tumors present a high grade of vascularisation. One patient showed high expression of Hif-1a, Fli-1 and Pax2 and high score for CD34 in intratumoral vessels.

1. A combination of antitumoral agents, wherein said antitumoral agents comprise ombrabulin, a taxane derivative and a platinum derivative, said ombrabulin, said taxane derivative and said platinum derivative each being in the form of a free base, of an addition salt with a pharmaceutically acceptable acid, of a hydrate, or of a solvate, wherein said combination is well tolerated, does not exacerbate the toxicity of each of the antitumoral agents, and allows the treatment of advanced solid tumors either by stabilizing or a tumor or by inducing a partial or a complete regression of a tumor.

2. The combination according to claim 1 wherein ombrabulin is in the form of the hydrochloride salt.

3. The combination according to claim 1 wherein the taxane derivative is chosen from paclitaxel and docetaxel.

4. The combination according to claim 1 wherein the platinum derivative is chosen from cisplatin and carboplatin.

5. The combination according to claim 1 wherein ombrabulin is in combination with docetaxel and cisplatin or in combination with paclitaxel and carboplatin.

6. The combination according to claim 1 comprising an effective quantity of ombrabulin, an effective quantity of a taxane derivative and an effective quantity of a platinum derivative.

7. The combination according to claim 6 wherein ombrabulin is administered at a dose of between 15 and 35 mg/m².

8. The combination according to claim 7 wherein ombrabulin is administered at a dose chosen from 15.5, 20, 25, 30 and 35 mg/m².

9. The combination according to claim 6 wherein the taxane derivative is docetaxel and is administered at a dose of 60 or 75 mg/m².

10. The combination according to claim 6 wherein the taxane derivative is paclitaxel and is administered at a dose of 175 or 200 mg/m².

11. The combination according to claim 6 wherein the platinum derivative is cisplatin and is administered at a dose of 75 mg/m².

12. The combination according to claim 6 wherein the platinum derivative is carboplatin and is administered at a dose of AUC 5 or 6.

13. The combination according to claim 5 wherein ombrabulin is in combination with docetaxel and cisplatin and wherein ombrabulin is administered at a dose of 20 mg/m², docetaxel is administered at a dose of 75 mg/m² and cisplatin is administered at a dose of 75 mg/m².

14. The combination according to claim 5 wherein ombrabulin is in combination with docetaxel and cisplatin and wherein ombrabulin is administered at a dose of 35 mg/m², docetaxel is administered at a dose of 75 mg/m² and cisplatin is administered at a dose of 75 mg/m².

15. The combination according to claim 5 wherein ombrabulin is in combination with paclitaxel and carboplatin and wherein ombrabulin is administered at a dose of 35 mg/m², paclitaxel is administered at a dose of 175 mg/m² and carboplatin is administered at a dose of 5 AUC.

16. The combination according to claim 5 wherein ombrabulin is in combination with paclitaxel and carboplatin and wherein ombrabulin is administered at a dose of 35 mg/m², paclitaxel is administered at a dose of 200 mg/m² and carboplatin is administered at a dose of 6 AUC.

17. The combination according to claim 1, wherein said treatment of advanced solid tumors comprises cyclic administration of said antitumoral agents and wherein each said cycle comprises an interval between two administrations of three weeks.

18. An article of manufacture comprising:
a packaging material;
an antitumoral pharmaceutical combination comprising ombrabulin, a taxane derivative and a platinum derivative, each of said ombrabulin, said taxane derivative and said platinum derivative being in the form of a free base, or of an addition salt with a pharmaceutically acceptable acid, or of a hydrate or solvate; and
a label or package insert contained within said packaging material indicating that said antitumoral pharmaceutical combination is administered to the patient at a recommended dose, and in a plurality of subsequent doses at a recommended dose separated in time from each other by three weeks.

19. The article of manufacture according to claim 18 wherein ombrabulin is in combination with docetaxel and cisplatin or in combination with paclitaxel and carboplatin.

20. The article of manufacture according to claim 19 wherein said packaging material indicates that said ombrabulin is administered at a dose of between 15 and 35 mg/m².

21. The article of manufacture according to claim 20 wherein said packaging material indicates that said ombrabulin is administered at a dose chosen from 15.5, 20, 25, 30 and 35 mg/m².

22. The article of manufacture according to claim 20 wherein said packaging material indicates that said taxane derivative is docetaxel and that it is administered at a dose of 60 or 75 mg/m².

23. The article of manufacture according to claim 20 wherein said packaging material indicates that said taxane derivative is paclitaxel and that it is administered at a dose of 175 or 200 mg/m².

24. The article of manufacture according to claim 20 wherein said packaging material indicates that said platinum derivative is cisplatin and that it is administered at a dose of 75 mg/m².

25. The article of manufacture according to claim 20 wherein said packaging material indicates that said platinum derivative is carboplatin and that it is administered at a dose of AUC 5 or 6.

26. The article of manufacture according to claim 20 wherein said ombrabulin is in combination with docetaxel and cisplatin and wherein said packaging material indicates that said ombrabulin is administered at a dose of 20 mg/m², said docetaxel is administered at a dose of 75 mg/m² and said cisplatin is administered at a dose of 75 mg/m².

27. The article of manufacture according to claim 20 wherein said ombrabulin is in combination with docetaxel and cisplatin and wherein said packaging material indicates that said ombrabulin is administered at a dose of 35 mg/m², said docetaxel is administered at a dose of 75 mg/m² and said cisplatin is administered at a dose of 75 mg/m².

28. The article of manufacture according to claim 20 wherein said ombrabulin is in combination with paclitaxel and carboplatin and wherein said packaging material indicates that said ombrabulin is administered at a dose of 35 mg/m², said paclitaxel is administered at a dose of 175 mg/m² and said carboplatin is administered at a dose of 5 AUC.

29. The article of manufacture according to claim 20 wherein said ombrabulin is in combination with paclitaxel and carboplatin and wherein said packaging material indicates that said ombrabulin is administered at a dose of 35 mg/m², said paclitaxel is administered at a dose of 200 mg/m² and said carboplatin is administered at a dose of 6 AUC.
